(19) United States
(12) Patent Application Publication
(10) Pub. No.: US 2019/0241507 A1
(43) Pub. Date: Aug. 8, 2019

(54) A Process for Manufacturing Isocyanates and/or Polycarbonates

(71) Applicant: Huntsman International LLC, The Woodlands, TX (US)

(72) Inventors: Jaco Meindert Van Der Leeden, The Hague (NL); Peter Muller, Hellevoetsluis (NL); Robert Henry Carr, Bertem (BE); Arend Jan Zeeuw, Wassenaar (NL)

(21) Appl. No.: 16/093,792

(22) PCT Filed: Apr. 24, 2017
(86) PCT No.: PCT/US2017/059612

§ 371 Date: Oct. 15, 2018

(30) Foreign Application Priority Data
May 10, 2016 (EP) 16168911.2

(51) Int. Cl. C07C 263/10; C07C 265/04; C07C 68/02; C07C 69/86

(52) U.S. Cl. CPC C07C 263/10; C07C 265/04; C08G 64/02; C07C 69/86; C07C 68/02

(57) ABSTRACT

A process for manufacturing isocyanates or polycarbonates comprising the steps of: providing a chlorine stream and carbon monoxide stream; reacting said chlorine stream and said carbon monoxide stream for providing a phosgene stream; cooling the phosgene stream to a temperature at which the phosgene in the phosgene stream is liquid, preferably, to a temperature that is 4° C. less or more than 4° C. less than the boiling point of phosgene, to form a liquid phosgene stream and a gas stream; separating the gas stream and the liquid phosgene stream; removing residual chlorine from the liquid phosgene stream to form a chlorine depleted phosgene stream and reacting the chlorine depleted phosgene stream to form an isocyanate or a polycarbonate.

The present invention is related to a new process for making isocyanates and or polycarbonates by preparing phosgene that can provide isocyanates or polycarbonates that are light coloured, and the use of the isocyanates in urethane compounds, such as polyurethane foams.

Isocyanates and isocyanate mixtures are prepared by phosgenation of corresponding amines. For polyurethane foams, use is made, for example, of bifunctional or polyfunctional aromatic isocyanates of the diphenylmethane diisocyanate series (MDI). The preparation process of such isocyanates, the phosgenation and subsequent work-up (removal of the solvent; separation of monomeric MDI) often results in dark-coloured products which in turn give yellowish polyurethane foams or other, likewise discoloured polyurethane materials. This is undesirable, since such discoloration adversely affects the overall visual impression and allows slight inhomogeneities to be observed. Light-coloured isocyanates or isocyanates which contain a reduced amount of colour-imparting components are therefore preferred as raw materials.

One of the reasons why dark-coloured isocyanates are formed is because of the quality of the phosgene used to phosgenate the amine compound. The phosgene used to convert amines to the corresponding isocyanates is manufactured conventionally at industrial scale by reacting chlorine (here and henceforth meaning dichlorine or Cl₂) with carbon monoxide using customary and well known processes. The phosgene manufacturing is carried out typically over one or more generally high purity carbon catalysts known in the art which may have been optionally surface- or otherwise treated. It is well known that commercial carbon catalysts can be made more active by means of an initial activation treatment with chlorine. When free unreacted chlorine enters together with phosgene in an isocyanate manufacturing system, significant levels of undesirable by-products are formed. For this reason, usually when phosgene is made that will be used for making isocyanates, a stoichiometric excess of CO is used. Unreacted CO can be separated off, optionally purified and returned to the phosgene plant.

To improve the quality of the phosgene, US20040024244 teaches that when chlorine is used having low bromine content of less than 50 ppm, phosgene is made that provides light coloured isocyanates when reacted with the corresponding amine. On the other hand, WO2010060773 teaches that when using a mole ratio carbon monoxide over chlorine that is slightly above stoichiometric, such as less than or equal to 1.025:1.000 and above 1.000:1.000, it is possible to use chlorine having higher amount of bromine, which may be between 50 and 500 ppm, and which phosgene can be used to provide light coloured isocyanates. As described, an excess of CO is preferred for making phosgene so that all the chlorine reacts away, however when using a small excess, there is a risk that a considerable amount of chlorine is not reacted, and can enter the isocyanate plant to form undesirable side-products. For this reason, there remains further a need for a process for making phosgene that can be used for isocyanates, which phosgene has no deleterious effect on the colour of the isocyanates formed, taking into account that the amount of chlorine entering the phosgenation reactor together with the phosgene stream, is reduced to a minimum.

Also for the production of polycarbonates, phosgene is often used as a raw material. It is known that carbontetrachloride, an impurity that is often formed when making phosgene, can form organic chlorides as impurities in the production of polycarbonates and high levels of these organic chlorides impact the polymerization reaction and result in an adverse colour as also described in patent application with the publication number WO 2015/119982. Further, it is also described that also the bromine molecules present in the phosgene can have a bad influence on the colour of the polycarbonates (see e.g. JP 2012/254895 and JP2010/195641).

Therefore, it is an object of the invention to provide a process for making isocyanates or polycarbonates where the used phosgene does not contribute to dark coloured isocyanates or to the colour of polycarbonates, and/or where no or a minimum amount of chlorine enters the phosgenation reaction.

This object, amongst others, is met, at least partially by a process according to claim 1 or 2.

In particular, this object, amongst others, is met, by a process for manufacturing isocyanates comprising the steps of:
a) providing a chlorine stream and carbon monoxide stream, wherein the chlorine stream comprises less than 500 ppm bromine, preferably between 50 and 500 ppm bromine;
b) reacting said chlorine stream and said carbon monoxide stream for providing a phosgene stream, wherein the mole ratio carbon monoxide in the carbon monoxide stream over chlorine in the chlorine stream is in a range of between 0.900:1.000 to 1.025:1000;
c) cooling the phosgene stream to a temperature at which the phosgene in the phosgene stream is liquid, preferably, to a temperature that is 4° C. less or more than 4° C. less than the boiling point of phosgene, to form a liquid phosgene stream and a gas stream;
d) separating the gas stream and the liquid phosgene stream;
e) removing residual chlorine from the liquid phosgene stream to form a chlorine depleted phosgene stream;
g1) reacting the chlorine depleted phosgene stream with an amine compound to form a corresponding isocyanate compound.

In particular, this object, amongst others, is also met, by a process for preparing polycarbonate compounds comprising the steps of:
a) providing a chlorine stream and carbon monoxide stream, wherein the chlorine stream comprises less than 500 ppm bromine, preferably between 50 and 500 ppm bromine;
b) reacting said chlorine stream and said carbon monoxide stream for providing a phosgene stream, wherein the mole ratio carbon monoxide in the carbon monoxide stream over chlorine in the chlorine stream is in a range of between 0.900:1.000 to 1.025:1000;
c) cooling the phosgene stream to a temperature at which the phosgene in the phosgene stream is liquid, preferably, to a temperature that is 4° C. less or more than 4° C. less than the boiling point of phosgene, to form a liquid phosgene stream and a gas stream;
d) separating the gas stream and the liquid phosgene stream;
e) removing residual chlorine from the liquid phosgene stream to form a chlorine depleted phosgene stream;
g2) reacting the chlorine depleted phosgene stream to form a polycarbonate compound.

The inventors surprisingly found that in such a process it is possible to use chlorine from which the bromine does not need to be first removed by a purification stage, and this chlorine can be used to make phosgene that by reacting with an amine compound produces light coloured isocyanates. Also, the chlorine depleted phosgene stream can be used to produce light coloured polycarbonates. Further, it is found that by using the process of the invention it is possible to use a much smaller excess of CO, or even a small molar excess of Cl₂ for making the phosgene in a first reactor. Furthermore, catalysts used for making the phosgene can be used longer. Often catalysts are renewed from the moment too much chlorine remains unreacted. Using the process according to this invention, the catalyst can be used longer, since unreacted chlorine is separated later in the process. The process of the invention is able to provide phosgene that can be used to make light coloured isocyanates and/or light coloured polycarbonates.

The inventors found that when a too high excess of CO is used, the bromine that is present in the chlorine reacts with the CO and forms bromophosgene compounds (i.e. dibromophosgene COBr₂ or monobromophosgene COBrCl). Without being limited by theory, it is thought that these bromophosgene compounds contribute to the formation of dark coloured isocyanate or polycarbonate products. When making phosgene using a stoichiometrical amount of CO and Cl₂ or a small excess of CO, the bromophosgene compounds are not or almost not formed. When only a small excess of CO is used, a stoichiometric amount of CO is used or a small excess of Cl₂ is used in a first reactor, no or only a very little amount of COBrCl is present in the resulting phosgene stream. Most of the Br₂ or BrCl originally present in the chlorine can be in similar form in the phosgene stream. It is thought that these molecules, when present in the phosgene, do not contribute significantly to the overall colour of the isocyanates or the polycarbonates formed using that phosgene. The invention now provides a process wherein after a phosgene stream is formed, this stream is cooled down to a temperature at which the phosgene is liquid and where dibromine and bromine monochloride predominantly dissolve in the phosgene stream. At such temperature, CO will remain predominantly in the gas phase and can easily be removed. Also a part of the chlorine remains in the gas phase. Another part dissolves in the phosgene. This depends on the temperature and pressure that are used at the moment that the phosgene is cooled down below its boiling point. The chlorine present in the liquid phosgene fluid stream is removed later in the process. The Br₂ and BrCl remain in the liquid phosgene fluid stream when the chlorine is removed from the phosgene. The chlorine depleted phosgene stream can then be used for making isocyanates by reacting with an amine compound. Since no bromophosgene compounds are present, the phosgene does not contribute to dark coloured isocyanates. The chlorine depleted phosgene stream can also be used to produce carbonates such as diaryl carbonate, dialkyl carbonate and polycarbonate, and helps in improving the colour properties of the carbonates.

The mole ratio carbon monoxide in the carbon monoxide stream over chlorine in the chlorine stream in step b) is in a range of between 0.900:1.000 to 1.025:1.000. This way no or almost no COBrCl is formed. When stoichiometrically more chlorine than carbon monoxide is used, then the excess chlorine will end up in the gaseous stream or will be removed from the phosgene fluid stream. When stoichiometrically more CO is used, most of the chlorine is reacted away. The unreacted CO will end up in the gas stream. This CO can be used for other purposes, such as recycling to step a, or can be used in another reactor for making phosgene.

The molar ratio of carbon monoxide in the carbon monoxide stream over chlorine in the chlorine stream in step b) can be controlled and adjusted during operation of the process according to the invention. The adjustment may be done e.g. by changing the relative flow rate of carbon monoxide in the carbon monoxide stream in view of the chlorine in the chlorine stream or by changing the pressure in the carbon monoxide stream or chlorine stream, or both.

Means for controlling the process by use of on-line analysers for carbon monoxide and halogens or on-line or off-line determination of total chlorine or total bromine in the product that is made using the phosgene such as isocyanate or polycarbonate, may be applied. Controlling the process may include calculating the amount or content of carbon monoxide and/or the amount or content of chlorine in various fluid streams, and calculating the molar ratio of carbon monoxide and chlorine, based upon calculated or measured values of process parameters and settings, which parameters and/or settings are provided from the process to manufacture the product that is made using the phosgene such as isocyanates or the polycarbonates.

As described, the formed phosgene stream will be cooled down to a temperature at which the phosgene in the phosgene stream is liquid. The temperature can vary depending on the pressure that is used. As e.g., at 1 barg, phosgene is liquid at a temperature below 8.3° C. which is the boiling point of phosgene at 1 barg. A person skilled in the art knows that at a higher pressure, the boiling point is higher. At this temperature the bromine species are dissolved in the phosgene. Preferably, the temperature is 4° C. less or more than 4° C. less than the boiling point of phosgene. This is preferred, because at such temperature the BrCl, having a boiling point of 5° C. at 1 barg, is also in its liquid form. Br₂ is liquid at the boiling point of phosgene. The temperature must be higher than the boiling point of carbon monoxide. The temperature can be below than, equal to or higher than the boiling point of chlorine (the boiling point of chlorine at 1 barg is −34° C. and is higher at higher pressures). When the temperature is higher than the boiling point of chlorine, the chlorine is in the gas form and at least part of the chlorine will end up in the gas stream, but also a considerable amount of chlorine can be present and can be dissolved in the liquid phosgene fluid stream. When the temperature is below the boiling point of chlorine, most of the chlorine will end up in the liquid phosgene stream. A skilled person knows that the temperature and pressure of several streams play a role in the concentration of the compounds that will end up in several streams. Further, a skilled person knows that the operating conditions can be adjusted to optimize the concentrations of the compounds in the streams.

The chlorine dissolved in the phosgene fluid stream is later removed from the phosgene.

Since chlorine can be removed in a later step, it is also possible that the phosgene stream is cooled down to a temperature that is lower than the boiling point of chlorine.

When higher pressure is used, higher temperatures can be used for making the liquid phosgene fluid stream. Examples of pressures and temperatures at which the phosgene stream can be made liquid are e.g. −20° C. at 3 bar. At this temperature, the chlorine is liquid and most of the chlorine will end up in the liquid phosgene stream. Another possible temperature that can be used is 10° C. at 3 bar, where the chlorine is in the gas phase and most of this chlorine will end up in the gas stream. The vapor and liquid streams can be monitored via inline analyzing via means known by a person skilled in the art such as UV/Vis and Infra red spectroscopy. It is possible to optimize the conditions of the streams, e.g. by changing the pressure, volume, temperature, and/or flow rates. Also depending on what the target solution one wants to obtain, the conditions can be changed. E.g. the process can be designed in a way that the gas stream comprises less or more phosgene. Cooling down the phosgene stream can be done by any known cooling means in the art. This can for example be by process chillers, air coolers, water coolers, chillers and/or any combination thereof.

When the phosgene stream is cooled down, a liquid phosgene fluid stream and a gas stream are separated. The gas stream can e.g. be removed at the top of the cooler means as vent gasses. The gas stream mainly comprises CO, Cl₂, N₂, Ar, CO₂ and phosgene. The gas stream comprises substantially no bromine species.

In one embodiment, the process of the invention further comprises a step f) wherein the separated gas stream from step d) is brought to a second reactor, that is optionally cooled, where the chlorine and carbon monoxide present in the separated gas stream react to form a second phosgene stream. In this phosgene reactor, substantially no bromine is present, since the bromine species reside in the liquid phosgene fluid stream separated off in step d. To this second phosgene reactor also extra carbon monoxide can be added to make sure that there is an excess of CO and all the chlorine species can react away with the CO to form the second phosgene stream. This second phosgene stream can be used for making isocyanates, polycarbonates, or can be used for other purposes. In factories where phosgene is made as raw material for both isocyanates and polycarbonates, it can be an advantage to use this second phosgene reactor for making phosgene that can be used in polycarbonates. This way process requirements for limiting the formation of carbon tetrachloride by-product can be used in connection with this second phosgene reactor. Such requirements are known by a person skilled in the art and are e.g. described in the patent application WO 2015/119982. The chlorine depleted phosgene stream from step e) can then be used for making isocyanates.

After separation from the gaseous stream, the liquid phosgene fluid stream flows to another column that is designed so that the bromine species ClBr and Br₂ pass through the column but residual Cl₂ that is still present in the liquid phosgene fluid stream in step e) is removed. This removal can be done by any known means in the art. The phosgene fluid stream can be in its liquid form or gas form. The means to remove the chlorine from the phosgene fluid stream depend on the phase of the stream. In one embodiment, the chlorine is removed from the phosgene fluid stream in its liquid phase by stripping the liquid phosgene stream with a suitable gas. This gas can e.g. be CO, N₂, CO₂. Preferably CO is used. The stripping of the chlorine can be done at or around the boiling point of chlorine. This way the chlorine can easily flow together with the gas flow. A person skilled in the art knows that the stripping column is designed in a way that substantially all the chlorine is removed from the phosgene fluid stream. The design also depends on the temperature and pressure conditions that will be used for removing the chlorine. Also other means that are able to separate chlorine from a fluid comprising chlorine and phosgene can be used such as using methods based on membranes [semi-permeable membranes for gas separations, membrane contactor units, and the like]. This way a chlorine depleted phosgene stream is formed.

The removed chlorine can then flow back to the chlorine stream of step a) to be used to make phosgene. It can also flow to the second phosgene reactor, although the latter is not preferred, since residual bromine species might have been removed in step d) together with the chlorine. When the chlorine is returned back to the chlorine stream of step a), the residual bromine species can be removed again according to the process of the invention.

Both the chlorine and the carbon monoxide may be provided as fresh raw streams of material, or may be partially provided as recycled material. The chlorine may be provided or partly provided from a chlorine-forming process which uses HCl from an isocyanate production process or polycarbonate process, or can be produced from salt sea water or other salt water or brine source, preferably after purification, or any other process as is well known in the art. It is clear that adjustments of flows of raw material or optionally streams of recycled materials may be done in any known way which is well known in the art of conducting chemical processes, e.g. by manual interventions, e.g. for adjustment of appropriate valve settings, or by adjusting flows in a controlled way by means of control software in combination with automated valves controlled by said control software.

The chlorine stream comprises bromine. The bromine content in the chlorine stream may be up to 500 ppm and can be in the range of 50 to 500 ppm.

The chlorine depleted phosgene stream can then be used in a next step as raw material for making isocyanates or polycarbonates.

In one embodiment the amine compound can be any kind of primary amine compound, which can react appropriately with phosgene to give isocyanates. Suitable amines are, in principle, all linear or branched, saturated or unsaturated aliphatic or cycloaliphatic or aromatic primary monoamines or polyamines, provided that these can be converted into isocyanates by means of phosgene. Examples of suitable amines are 1,3-propylenediamine, 1,4-butylenediamine, 1,5-pentamethylenediamine, 1,6-hexamethylenediamine and the corresponding higher homologues of this series, isophoronediamine (IPDA), cyclohexyldiamine, cyclohexylamine, aniline, phenylenediamine, p-toluidine, 1,5-naphthylenediamine, 2,4- or 2,6-toluenediamine or a mixture thereof, 4,4′-, 2,4′- or 2,2′-diphenylmethanediamine or mixtures thereof and also higher molecular weight isomeric, oligomeric or polymeric derivatives of the abovementioned amines and polyamines. In a preferred embodiment of the present invention, the amine used is an amine of the diphenylmethanediamine series or a mixture of two or more such amines.

After reacting with the chlorine depleted phosgene stream, the abovementioned compounds are in the form of the corresponding isocyanates, e.g. 1,3-propylenediisocyanate; 1,4 butylenediisocyanate; 1,5-pentamethylenediisocyanate; hexamethylene 1,6-diisocyanate, isophorone diisocyanate, cyclohexyl isocyanate, cyclohexyl diisocyanate, phenyl isocyanate, phenylene diisocyanate, 4-tolyl isocyanate, naphthylene 1,5-diisocyanate, tolylene 2,4- or 2,6-diisocyanate or mixtures thereof, diphenylmethane 4,4′-, 2,4′- or 2,2′-diisocyanate or mixtures of two or more thereof, or else higher molecular weight oligomeric or polymeric derivatives of the abovementioned isocyanates or as mixtures of two or more of the abovementioned isocyanates or isocyanate mixtures.

In a preferred embodiment of the present invention, the amines used are the isomeric, primary diphenylmethane-diamines (MDA) or their oligomeric or polymeric derivatives, i.e. the amines of the diphenylmethanediamine series. Diphenylmethanediamine, its oligomers or polymers are obtained, for example, by condensation of aniline with formaldehyde. Such oligoamines or polyamines or mixtures thereof are also used in a preferred embodiment of the invention.

The reaction of the phosgene with one of the abovementioned amines or a mixture of two or more of such amines can be carried out continuously or batchwise in one or more stages. If a single-stage reaction is carried out, this reaction preferably takes place at a temperature from about 60 to 200° C., for example from about 130 to 180° C.

The phosgenation reaction can, for example, be carried out in two stages. Here, in a first stage, the reaction of the phosgene with the amine or the mixture of two or more amines is carried out at a temperature from about 0 to about 130° C., for example from about 20 to about 110° C., or from about 40 to about 70° C., with a time of from about 1 minute to about 2 hours being allowed for the reaction between amine and phosgene. Subsequently, in a second stage, the temperature is increased to from about 60 to about 190° C., in particular from about 70 to 170° C., over a period of, for example, from about 1 minute to about 5 hours, preferably over a period of from about 1 minute to about 3 hours. In a preferred embodiment of the invention, the reaction is carried out in two stages.

Alternatively, more stages may be defined according to temperature/pressure/reaction time parameters and the like, such stages being carried out in one or more vessels operated in batch, continuous or semi-batch modes. Gas phase processes are also known for making isocyanates.

During the phosgenation reaction, superatmospheric pressure can, in a further preferred embodiment of the invention, be applied, for example up to about 100 bar or less, preferably from about 1 bar to about 50 bar or from about 2 bar to about 25 bar or from about 3 bar to about 12 bar. However, the reaction can also be carried out under atmospheric pressure or at a pressure below ambient pressure.

Excess phosgene is preferably removed at a temperature from about 50 to 180° C. after the reaction. Preferably the excess phosgene is removed at a temperature from about 50° C. to 130° C. At lower temperatures within the specified range, a better color of the end product can be obtained. The removal of remaining traces of solvent is preferably carried out under reduced pressure, for example the pressure should be about 500 mbar or less, preferably less than 100 mbar. In general, the various components are separated off in the order of their boiling points; it is also possible to separate off mixtures of various components in a single process step.

According to some embodiments of the present invention, the amine compound may comprise diaminodiphenylmethane. Diaminodiphenylmethane may also be referred to as DADPM or MDA. The amine compound may even substantially consist of a mixture of isomers of diaminodiphenylmethane, such as 4,4′-MDA, 2,4′-MDA in combination with higher oligomers or homologues of MDA.

Phosgenation of a base product comprising diaminodiphenylmethane, i.e. isomers or homologues of MDA, results in a polyisocyanate mixture comprising methylene diphenyl diisocyanate (MDI), typically a mixture of isomers of MDI, e.g. such as 4,4′-MDI, 2,4′-MDI, and homologues of MDI or oligomeric polyisocyanates. This resulting polyisocyanate mixture is often referred to as polymeric MDI, or PMDI.

Since in the phosgenation reaction no COBrCl compounds are available, the phosgene did not contribute significantly to the formation of dark coloured isocyanates. The colour of the produced isocyanate may be characterized by using in-line or off-line techniques. The measured colour can be quoted in terms of the various “colour space” systems such as Hunterlab Lab and CIE L*a*b* and can be determined either on the original isocyanate material or on a solution of the isocyanate in a suitable solvent. Quoting isocyanate colour in the Hunterlab Lab colour space or system, the isocyanate as provided by the process, i.e. not brought in solution, may have a colour grade/value of L greater than 30, preferably greater than 35, more preferred greater than 40, still preferably greater than 45.

According to some embodiments of the present invention, the colour of the isocyanate obtained by the process according to the present invention may have a Hunterlab Lab colour grade/value L larger than 30. Changes in a or b parameters of the Hunterlab Lab space determined on the isocyanate product may also arise as a result of the present invention and may be beneficial in some applications.

For measuring colour grades in HunterLab colour space or the CIE L*a*b* colour space, typically HunterLab test equipment is used, as is well known in the art.

According to some embodiments of the process of the present invention, the isocyanate obtained may comprise 30 to 500 ppm of bromine in bound form, such as 30 to 150 ppm of bromine in bound form, e.g. 50 to 150 ppm bromine in bound form.

According to some embodiments, the isocyanate may have a colour having a Hunterlab Lab grade/value L larger than 30.

According to a further aspect of the present invention, an isocyanate obtained by the process described above may be used for providing polyurethane, such as e.g. rigid or flexible polyurethane foam, polyurethane coatings, adhesives, polyisocyanurate polyurethane based products and to bind other materials together, such as wood-based products, and the like.

As described the chlorine depleted phosgene stream is reacted with at least one amine compound (i.e. phosgenation of an amine), providing an isocyanate. After phosgenation of the amine, some CO also may leave the plant with the hydrogen chloride gas which is typically then used in one or more further chemical processes (“exported”). The compositions of the carbon monoxide, optionally both the fresh carbon monoxide and the carbon monoxide recycled from after production of the phosgene, chlorine, phosgene, export-HCl and recycle gas streams can be monitored by means of on-line analytical techniques such as gas chromatography, mass spectrometry or spectroscopic techniques (UV-Vis, IR, NIR, etc).

Control of the operation of the phosgene plant, i.e. the production of phosgene, and the subsequent production of isocyanate by phosgenation of a corresponding amine, in terms of achieving the desired ratios of feed gas streams, can be carried out by manual intervention or by means of control software and corresponding valving systems, and can optionally include inputs based on isocyanate product composition, such as MDI product composition, as well as on composition and/or volume of one or more of the various gas streams.

In an embodiment of the invention the reaction of the amine or the mixture of two or more amines with the phosgene, is carried out in a solvent or a mixture of two or more solvents. As solvent, it is possible to use all solvents suitable for the preparation of isocyanates. These are preferably aromatic, aliphatic or alicyclic hydrocarbons or their halogenated derivatives. Examples of such solvents are aromatic compounds such as monochlorobenzene (MCB) or dichlorobenzene, for example o-dichlorobenzene, toluene, xylenes, naphthalene derivatives such as tetralin or decalin, alkanes having from about 5 to about 12 carbon atoms, e.g. hexane, heptane, octane, nonane or decane, cycloalkanes such as cyclohexane, esters and ethers such as ethyl acetate or butyl acetate, tetrahydrofuran, dioxane or diphenyl ether.

The invention is further illustrated by the following drawing.

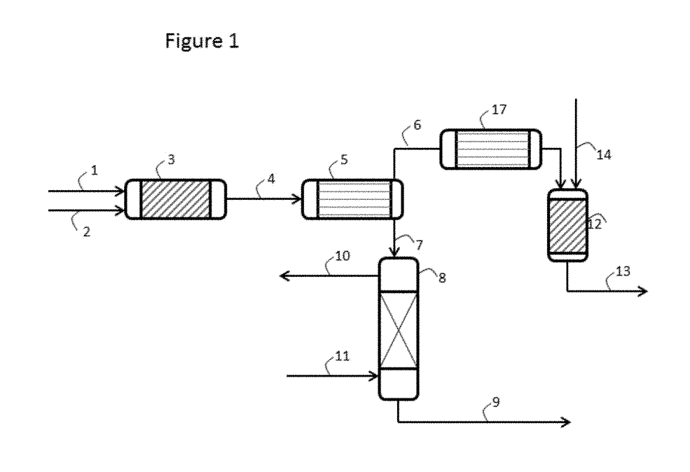

FIGS. 1, 2 and 3: Representations of a process flow for making a phosgene stream and separating the phosgene stream to provide a stream that can be used for making isocyanates according to the invention.

FIG. 1 represents a process flow wherein a carbon monoxide stream 1 and a chlorine stream 2 enter at least one reactor 3 to form a phosgene stream 4. These streams are gas streams. Reactor 3 may optionally be configured to enable generation of steam by making use of the exotherm of the phosgene-forming reaction, as is known in the art, the steam thus produced being therefore available as a heating source for other purposes. The phosgene stream 4 comprises unreacted carbon monoxide, chlorine, phosgene, bromine and bromine monochloride. The amount COBrCl is very low or even non existing due to specific mole ratio carbon monoxide in the carbon monoxide stream over chlorine in the chlorine stream that is used. The phosgene stream is brought to one or more heat exchangers or coolers 5, preferably a condenser, that cools down the gas stream to a temperature at which the phosgene is in its liquid phase. The liquid phosgene stream 7 is separated from the gas stream 6. The liquid phosgene stream now comprises all the bromine species. The liquid phosgene stream is then brought to at least one column 8 that is designed to remove the chlorine from the phosgene, e.g. a stripping column. Column 8 may have a reboiler and/or may have a condenser. The chlorine in the column can e.g. be removed by stripping with carbon monoxide 11 that enters at the bottom of the column 8. The chlorine leaves the column in a stream 10 comprising chlorine and the stripping gas e.g. CO and can at least partly be recycled to make phosgene via the chlorine stream 2, the CO stream 1 or can be fed directly to reactor 3.

The chlorine depleted phosgene stream 9 now comprises all bromine species and can be used to make isocyanates and/or polycarbonates. The gas stream 6 that is separated from the liquid phosgene stream 7, does not comprise bromine species. The gas stream 6 comprises chlorine, phosgene, carbon monoxide. This stream can be brought to at least one reactor 12, optionally via at least one heat exchanger 17. The reactor 12 is designed for making phosgene. The heat exchanger may be required to get the streams up to temperature again for the reaction in reactor 12. If required further carbon monoxide 14 can be added, which is required to make sure that an excess of carbon monoxide is present. The phosgene stream 13, can be used to make isocyanates and/or polycarbonates.

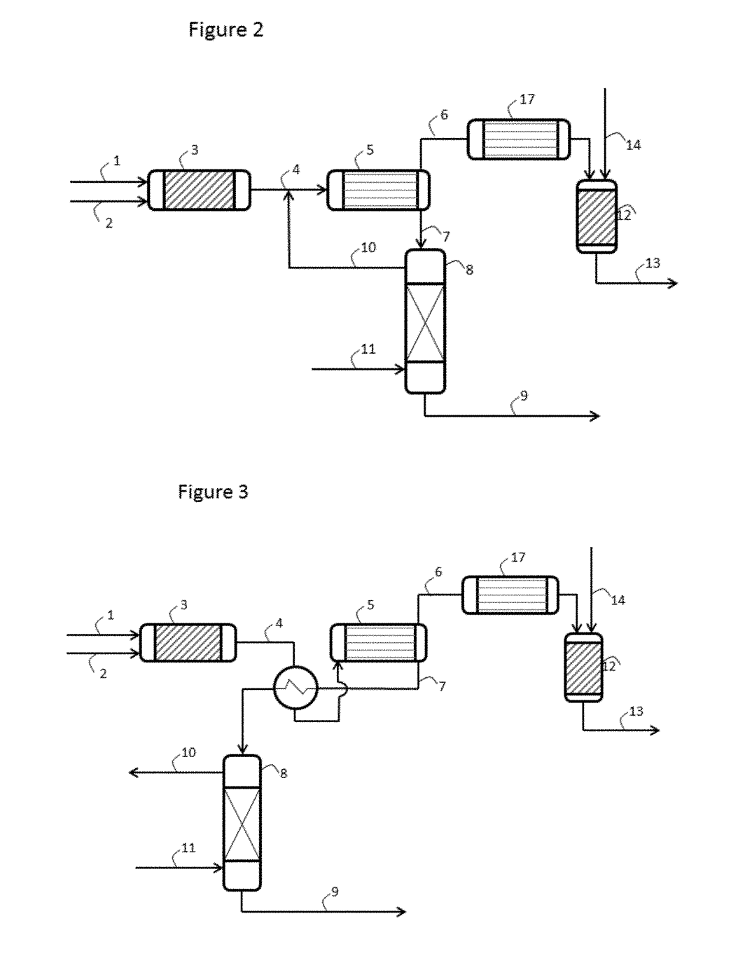

FIG. 2 is a drawing representing a flow scheme of another embodiment according to the invention wherein the streams are similar as described in FIG. 1 with the difference that stream 10 comprising chlorine and the stripping gas is fed to the at least one heat exchanger 5, directly or together with stream 4. Since stream 10 will mainly comprise the stripping gas, which is preferably CO, stripping can already occur in the at least one heat exchanger 5. The stripped chlorine gas will then separate from the liquid phase together with the other gasses in stream 6. In case that CO is used as stripping gas, it is possible that stream 14 is no longer required.

FIG. 3 is a drawing representing a flow scheme of another embodiment according to the invention wherein the streams are similar as described in FIG. 1 with the difference that stream 4 and 7 pass through a cross heat exchanger. This way the warm stream 4 coming from the phosgene reactor is able to warm up stream 7 which allows to perform the stripping in column 8 at a temperature that is higher than the temperature for cooling down in stream 5. This warmer temperature may facilitate the stripping in column 8.

1. A process for manufacturing isocyanates comprising the steps of:
a) providing a chlorine stream and carbon monoxide stream, wherein the chlorine stream comprises less than 500 ppm bromine;
b) reacting said chlorine stream and said carbon monoxide stream for providing a phosgene stream, wherein the mole ratio carbon monoxide in the carbon monoxide stream over chlorine in the chlorine stream is in a range of between 0.900:1.000 to 1.025:1000;
c) cooling the phosgene stream to a temperature at which the phosgene in the phosgene stream is liquid, to form a liquid phosgene stream and a gas stream;
d) separating the gas stream and the liquid phosgene stream;
e) removing residual chlorine from the liquid phosgene stream to form a chlorine depleted phosgene stream;
g1) reacting the chlorine depleted phosgene stream with an amine compound to form a corresponding isocyanate compound.

2. A process for preparing polycarbonate compounds comprising the steps of:
a) providing a chlorine stream and carbon monoxide stream, wherein the chlorine stream comprises less than 500 ppm bromine;
b) reacting said chlorine stream and said carbon monoxide stream for providing a phosgene stream, wherein the mole ratio carbon monoxide in the carbon monoxide stream over chlorine in the chlorine stream is in a range of between 0.900:1.000 to 1.025:1000;
c) cooling the phosgene stream to a temperature at which the phosgene in the phosgene stream is liquid, to form a liquid phosgene stream and a gas stream;
d) separating the gas stream and the liquid phosgene stream;
e) removing residual chlorine from the liquid phosgene stream to form a chlorine depleted phosgene stream;
g2) reacting the chlorine depleted phosgene stream to form a polycarbonate compound.

3. The process according to claim 1, further comprising the step f) bringing the separated gas stream from step d) to a second reactor and reacting chlorine and carbon monoxide present in the separated gas stream to form a second phosgene stream.

4. The process according to claim 3, wherein further carbon monoxide is provided to the second reactor.

5. The process according to claim 3, wherein the second phosgene stream flows to a reactor to react with an amine compound to form a corresponding polyisocyanate compound or is used to form a polycarbonate.

6. The process according to any one of the claims 1, wherein the removed residual chlorine of step e) flows back in the chlorine stream of step a).

7. The process according to claim 1, wherein the amine compound comprises diaminodiphenylmethane.

8. The process according to claim 1, wherein the colour of the isocyanate has a Hunterlab Lab colour grade/value L larger than 30.

9. The process according to claim 2, wherein the chlorine depleted phosgene stream reacts with a diol compound, preferably bisphenol A to form a polycarbonate compound.
